(19) United States
(12) Patent Application Publication
(10) Pub. No.: US 2004/0122086 A1
(43) Pub. Date: Jun. 24, 2004

(54) Method for the production of anhydrous, pharmaceutical formulations of xanthogenates

(76) Inventor: Eberhard Amtmann, Heidelberg (DE)

Correspondence Address: FOLEY AND LARDNER, SUITE 500, 3000 K STREET NW, WASHINGTON, DC, 20007, US

(21) Appl. No.: 10/474,424

(22) PCT Filed: Apr. 8, 2002
(86) PCT No.: PCT/EP02/03885

(30) Foreign Application Priority Data
Apr. 9, 2001 (DE) 101 17 728.3

(51) Int. Cl. A61K 31/28; A61K 31/265

(52) U.S. Cl. 514/492; 514/512

(57) ABSTRACT

The invention relates to anhydrous, pharmaceutical formulations of xanthogenates, to the production thereof and to agents containing said formulations for the treatment of virus, tumor or autoimmune diseases. The pharmaceutical preparations contain a xanthogenate of formula (I), wherein R₁ represents an optionally substituted aryl or alkyl radical and R₂ represents a metal atom, an optionally substituted alkyl, alkoxy, amino or ammonium group or halogen. Said preparations also contain an emulsifying agent that reduces the irritating effect of the xanthogenate and optionally an adjuvant enhancing the activity of the xanthogenate.

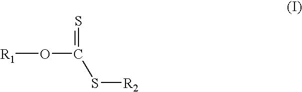

The invention relates to the preparation of pharmaceutical formulations of xanthates and compositions which contain these formulations, for the treatment of viral diseases, oncoses or autoimmune diseases.

Xanthates, in particular tricyclodecan-9-yl xanthate (D609) are known as substances having antiviral and antitumor activity, e.g. from “DNA and RNA virus species are inhibited by xanthates, a class of anti-viral compounds with unique properties” Sauer-G; Amtmann-E; Melber-K; Knapp-A; Muller-K; Hummel-K; Scherm-A, in Proc-Natl-Acad-Sci-U-S-A. 1984 June; 81(11): 3263-7; “Selective killing of tumor cells by xanthates” Amtmann-E; Sauer-G, in Cancer-Lett. 1987 June; 35(3): 237-44 and U.S. Pat. No. 4,602,037.

The antiviral properties of xanthates are evident, however, according to “Synergistic antiviral effect of xanthates and ionic detergents” Amtmann-E; Muller-Decker-K; Hoss-A; Schalasta-G; Doppler-C; Sauer-G, in Biochem-Pharmacol. 1987 May 1; 36(9): 1545-9 only in an acidified medium or in the presence of ionic detergents.

Since xanthates are acid-labile, their pharmaceutical use in acidified formulations is not practicable for stability reasons. Particularly effective additives have proven to be the potassium salts of decanoic, undecanoic and lauric acid. These additives have no antiviral properties themselves.

However, xanthates are strongly irritating. The pharmaceutical use of xanthates was therefore not possible until now. For D609, the maximum, tolerable concentration in ointments was found to be restricted to 1%. In animal experiments, it was shown that for an effective antiviral use at least 2.5% of D609 must be present in an ointment.

The pharmaceutical use of xanthates having antiviral and antitumor activity thus runs into three problems:

1. For the pharmacological activity, relatively high active compound concentrations are necessary. In animal experiments, for efficient inhibition of herpes simplex virus 1, concentrations of at least 2.5% of the active compound were needed.

2. Xanthates are unstable in the aqueous medium, in particular under slightly acidic conditions, such as prevail on the skin.

3. Xanthates have marked irritating properties. Use can therefore only be carried out in formulations containing at most 1% of active compound.

WO 96/14841 describes pharmaceutical formulations which contain a xanthate and an adjuvant increasing the activity of the xanthate incorporated in a steroid- or lipid-based carrier. These formulations are obtained by ultrasonic treatment of suspensions of the constituents in water. The presence of the adjuvant is described as indispensable for the incorporation of the xanthate.

A disadvantage of these formulations is especially the laborious preparation. The formulations also contain water.

We have now surprisingly found that by the simple addition of certain emulsifiers to xanthates their irritating action can be suppressed. The pharmaceutical use of the xanthates in therapeutically efficacious concentrations thereby becomes possible. Since these formulations are anhydrous, the chemical stability of the active compound is guaranteed over relatively long periods of time and thus pharmaceutical use is made possible. Using the formulations according to the invention, it is accordingly surprisingly possible to solve all three problems without laborious incorporation into a carrier. On the one hand, the tolerability is markedly improved. The antiviral efficiency is increased. An adequate chemical stability in aqueous medium is also guaranteed.

The present invention thus solves the above-mentioned problems by making available a process for the preparation of pharmaceutical formulations which contains a xanthate, if appropriate an adjuvant increasing its activity, and a steroid or phospholipid as an emulsifier which reduces the irritant action of the xanthate and of the adjuvant increasing the activity.

The preparation is carried out according to the invention in such a way that the xanthate and, if appropriate, the adjuvant is mixed with the emulsifier in substance, it preferably being possible to add a nonaqueous excipient, e.g. paraffin oil. Surprisingly, suspension in water and/or ultrasonic treatment is not necessary. The formulations obtained by simple mixing, for example by kneading or stirring, show the desired decreased irritant action and the necessary stability.

The formulation contains a xanthate of the general formula I

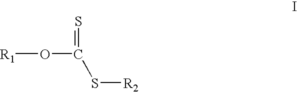

where R₁ is an optionally substituted aryl or alkyl radical. Preferably, R₁ is an adamantyl, norbornyl, tricyclodecyl, benzyl, linear or branched C₃-C₂₀-alkyl, C₃-C₂₀-cycloalkyl, furyl, pyridyl, anthracyl, naphthyl, phenanthryl, perinaphthyl, or quinuclidinyl radical, and the abovementioned linear or branched C₃-C₂₀-alkyl radical can be substituted by a hydroxyl-C₁-C₄-alkoxy group, a halogen atom or an amino group and the abovementioned C₃-C₂₀-cycloalkyl radical can likewise be substituted by a hydroxyl, C₁-C₄-alkoxy or C₁-C₄-alkyl group, a halogen atom or an amino group. Particularly advantageous for R₁ are cyclododecyl, dodecyl, undecyl, decyl, tricyclo[5,2,1,0^{2,6}] decyl, nonyl, octyl, bicyclo-[2,2,1]heptyl, cyclohexyl, hexyl, toluoyl radicals. A tricyclo [5,2,1,0^{2,6}] decyl radical is very particularly advantageous.

R₂ is a metal atom, an optionally substituted alkyl, alkoxy, amino or ammonium group or halogen. Preferably, R₂ is a monovalent or polyvalent metal atom, a linear C₁-C₆-alkyl radical, a C₁-C₆-alkyl radical substituted by hydroxyl, a C₁-C₆-alkoxy radical, an amino group, a C₁-C₆-alkamino radical, a (C₁-C₆-alkyl)₂-amino radical, a (C₁-C₆-alkyl)₃-ammonium radical, a halogen, 2,3-di-hydroxypropyl or hydroxy-(C₁-C₆-alkoxy)methyl. Sodium and potassium salts and also dimethylglycyl and methyl esters are particularly advantageous.

The adjuvant increasing the activity is preferably an ionic detergent. It can be a fatty acid having 6-19 C atoms or its salt. The potassium salts of decanoic, undecanoic or lauric acid are particularly advantageous.

The adjuvant increasing the activity can also be a sulfate having an aliphatic radical of 8-18 C atoms. Na laurylsulfate is particularly preferred.

Further, deoxycholic acid or a pharmaceutically tolerable salt thereof or a phosphonic acid is possible for the adjuvant.

Formulations in which, per one part of xanthate, 0.1 to 10 parts of adjuvant increasing the activity are contained have proven highly suitable. A ratio of xanthate to activity-increasing adjuvant of 1:1 is particularly advantageous.

The emulsifier reducing the irritant action is preferably a steroid. Stearylamine, cholesterol, cholestanol, cholanic acid, chondrillasterol and α,β,γ-sisterol, for example, are suitable. Cholesterol is particularly advantageous.

Suitable emulsifiers reducing the irritant action are also phospholipids, such as lecithin, phosphatidyl-choline, phosphatidylserine, phosphatidylinositol; glycolipids such as, for example, ganglioside, or sphingolipids, such as, for example, sphingomyelin. Lecithin, sphingomyelin and phosphatidylserine are particularly advantageous.

Preferably, 0.5 to 10 parts of emulsifier are employed per portion by weight of xanthate, in particular 2 to 6 parts.

A formulation is particularly preferred in which the emulsifier reducing the irritant action is cholesterol, the adjuvant increasing the activity is the Na or K salt of decanoic acid and the xanthate is tricyclo-[5,2,1,0^{2,6}]-9-yl xanthate. In particular, there is one part of potassium salt of decanoic acid and 4 parts of cholesterol to one part of xanthate.

A further particularly preferred formulation contains phosphatidylcholine as the emulsifier reducing the irritant action, the Na or K salt of decanoic acid as the adjuvant increasing the activity and tricyclo-[5,2,1,0^{2,6}]-9-yl xanthate as the xanthate. In particular, there is one part of decanoic acid and 4 parts of phosphatidylcholine to one part of xanthate.

The present invention furthermore makes available, as claimed in claims 15 to 20, compositions for the treatment of viral diseases, oncoses or autoimmune diseases, which contain the pharmaceutical formulation. The compositions further contain customary excipients. Other active compounds can also be contained if they adversely affect neither the action nor the stability of the xanthates.

Compositions in the form of ointments are particularly preferred, the ointment base used being a lipophilic substance. Petroleum jelly is preferably used as an ointment base. Concentrations of xanthate of 3% by weight have proven particularly suitable as a compromise between action and undesired irritation.

Anhydrous preparations for injection solutions are a further preferred embodiment. These preferably contain, in addition to the pharmaceutical formulation according to the invention, 0.1-5 parts of an alkali metal hydroxide, in particular KOH, per part of xanthate. The alkali metal hydroxide improves the solubility of the adjuvant and additionally stabilizes the xanthate in the injection solution.

The injection solution is obtained for use by dissolving or emulsifying in a physiologically tolerable medium, such as, for example, isotonic saline solution. In a particularly preferred embodiment, the xanthate-emulsifier mixture is made available in a kit containing the physiologically tolerable medium, the alkali metal hydroxide alternatively being present as a separate dry substance, in the xanthate-emulsifier mixture or dissolved in the physiologically tolerable medium.

The pharmaceutical formulations according to the invention and compositions which contain them are suitable for the treatment of viral diseases, oncoses and autoimmune diseases, such as, for example, herpes simplex and papilloma virus infections. The ointments according to the invention are particularly suitable for the treatment of skin diseases.

The following examples illustrate the invention further, but without restricting it.

### EXAMPLE 1

Preparation of an Ointment

For 100 g of ointment, 3 g of D609 are mixed with 12 g of cholesterol. In the course of this, 20-25 g of paraffin oil are added to facilitate the mixing of the powdered constituents. Petroleum jelly is added to the mixture to a total weight of 100 g and the whole is uniformly mixed in the mortar using a pestle. If the use of paraffin oil is dispensed with, the petroleum jelly content is to be increased correspondingly.

In the same manner, an ointment made of 3 g of D609 and 12 g of natural lecithin was prepared.

In the same manner, an ointment made of 3 g of D609 and 12 g of synthetic lecithin was prepared.

In the same manner, an ointment made of 3 g of D609 and 12 g of natural phosphatidylserine was prepared.

Greater amounts of ointments can be prepared analogously by known, industrial processes.

### EXAMPLE 2

Preparation of Injection Solutions

3 g of D609 and 3 g of potassium decanate are mixed with 12 g of natural or synthetic cholesterol. After this, 3 g of anhydrous KOH are added. The dry mixture is dissolved or emulsified by means of isotonic saline solutions immediately before injection. KOH can also be made available separately in dry form or as a solution or be the contents of a kit.

In the same manner, a mixture for an injection solution was prepared, cholesterol being replaced by the same amount of natural or synthetic lecithin.

In the same manner, a mixture for an injection solution was prepared, cholesterol being replaced by the same amount of natural or synthetic sphingomyelin.

In the same manner, a mixture for an injection solution was prepared, cholesterol being replaced by the same amount of natural or synthetic phosphatidylserine.

### EXAMPLE 3

Irritating Action of D609 in Phosphatidylcholine- or Cholesterol-Containing Ointments

D609 was mixed in a mortar together with the same proportion by weight of potassium salt of decanoic acid and two parts by weight of phosphatidylcholine or two or four parts by weight of cholesterol. After this, sufficient highly liquid paraffin oil and petroleum jelly (1 part by weight of paraffin oil, 4 parts by weight of petroleum jelly) were added such that in each case a concentration of 1.25; 2.5; 5.0; or 10% of D609 was achieved. In the same manner, D609-containing ointments without phosphatidylcholine or cholesterol were prepared.

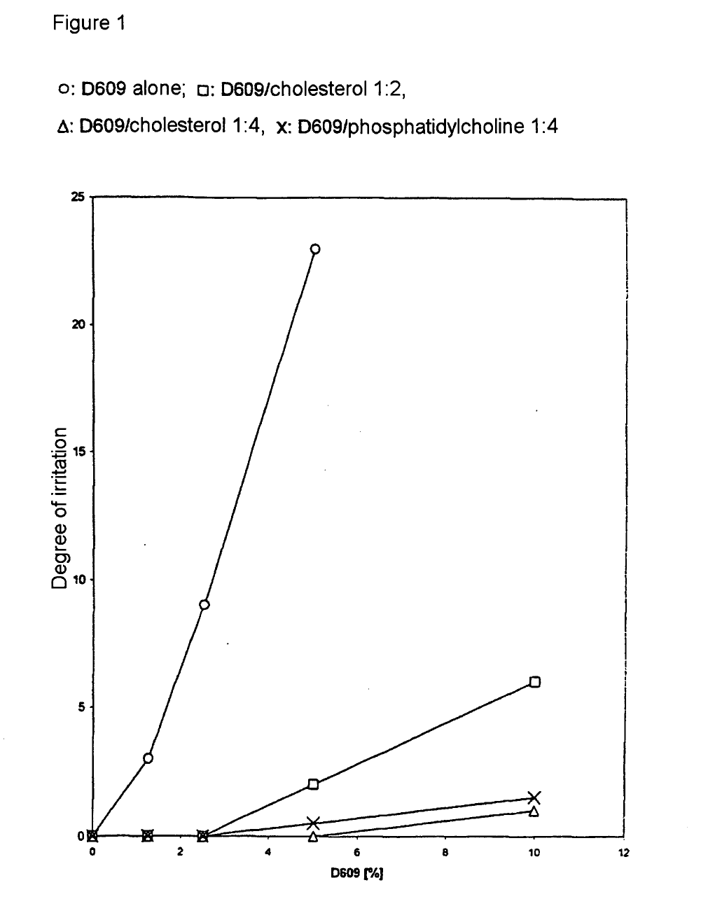

100 mg of ointment were applied twice in the neck region to 10 hairless mice each (strain Swiss CD3, Nu/Nu) at an interval of four hours. 12 hours after the last application, the degree of irritation was read off. The irritation was assessed according to the following scheme: slight erythema 0.5; severe erythema 1.0; ulceration 2.0. The assessments of all animals of a group were added and plotted in the graph (FIG. 1). As is seen, a marked irritation already occurs at a concentration of 2.5% of D609 in pure petroleum jelly. At 5%, this is very strongly pronounced. Ointments which contained two parts by weight of cholesterol showed a marked decrease in the symptoms. A significant irritation occurred only at 10% of D609. Ointments which contained 4 parts by weight of cholesterol were almost free of irritant action even at a concentration of 10% of D609. Only in one animal was a slight erythema observed. Ointments containing four parts by weight of phosphatidylcholine likewise showed a marked improvement in the tolerability. At 5% of D609, one animal showed a slight erythema, at 10% of D609 five animals.

### EXAMPLE 4

Antiviral Action of D609 in the Presence of Cholesterol

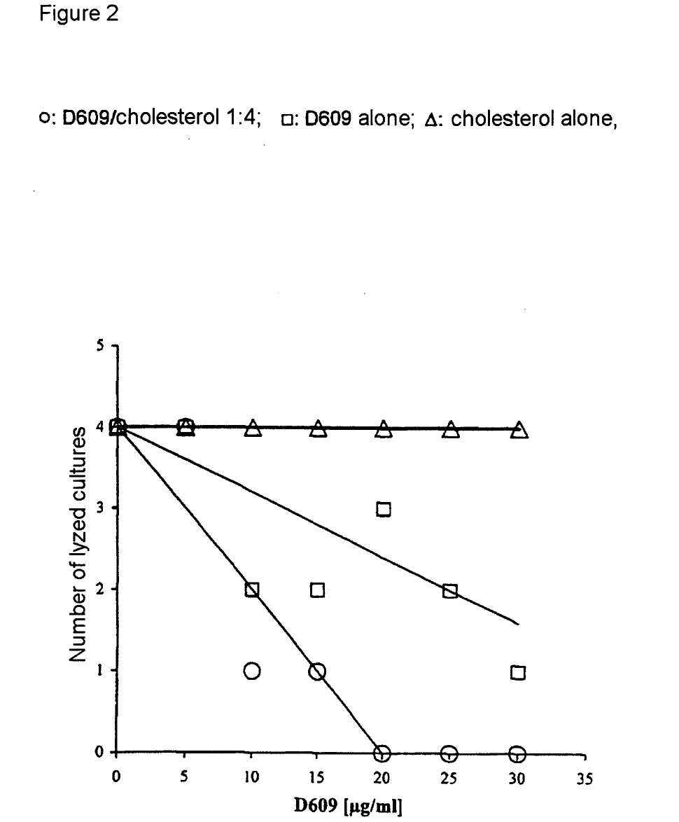

Monkey renal cells (Rita line) were infected in Linbro plates with 20 plaque-forming units of herpes simplex virus 1 (HSV 1), strain Angelotti. After 15 min, fresh cell culture medium (adjusted to pH 6.8 by reduction of the amount of sodium bicarbonate to 0.85 g/l and addition of 1 ml of 1 N HCl) which contained either 0, 5, 10, 15, 20, 25 or 30 μg/ml of D609 was added. All batches were carried out in quadruplicate. At the same time, cultures containing D609 and a fourfold concentration of cholesterol or containing cholesterol alone were treated. After 5 days, the monkey renal cells were lyzed in all untreated cultures by means of the cytopathogenic effect (cpe). After a further three days, the number of the cultures with cpe was determined for each D609 concentration. As can be inferred from FIG. 2, the number of cultures with cpe decreases with increasing D609 dose. The antiviral action of D609 was increased by cholesterol. While without cholesterol 25 μg/ml of D609 were necessary in order to reduce the number of the infected cultures by 50%, in the presence of cholesterol only 10 μg/ml of D609 were necessary. Cholesterol alone had no effect on the replication of HSV. Up to the highest concentration (120 μg/ml), all cultures were lyzed by HSV 1. The antiviral action of D609 is accordingly improved by more than a factor of 2.5 under conditions such as prevail on the skin (pH 6.8).

### EXAMPLE 5

Stability of D609 in Aqueous Solution in the Presence of Cholesterol

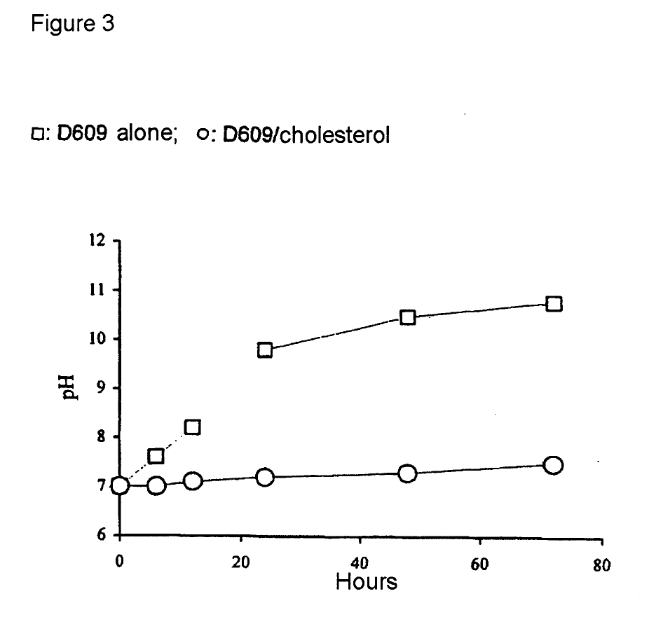

D609 was dissolved in distilled water in a concentration of 1 mg/ml. 100 ml of this solution in each case were filled into a 200 ml Erlenmeyer flask. One of two solutions was treated with 1 g of cholesterol. Both solutions were incubated at 37° C. The pH of the solutions was determined at regular intervals. D609 decomposes in the presence of water to give tricyclodecanol, carbon disulfide and KOH. The pH of the solution increases with the formation of KOH. The increase in the pH is therefore a measure of the decomposition of D609. It can be seen from FIG. 3 that D609 shows marked decomposition in aqueous solution as early as within 6 hours. In the presence of cholesterol, even after 72 hours only a small pH increase and thus decomposition is to be observed.

This example shows that by simple mixing of the xanthate with the emulsifier a stabilization is achieved even in aqueous solution. No ultrasonic treatment is necessary.

1. A process for the preparation of an anhydrous, pharmaceutical formulation, comprising a xanthate of the formula I 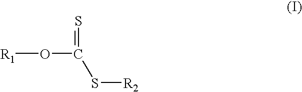
where R₁ is an optionally substituted aryl or alkyl radical and
R₂ is a metal atom, an optionally substituted alkyl, alkoxy, amino or ammonium group or halogen, and an emulsifier and, if appropriate, an adjuvant increasing the activity of the xanthate,
characterized in that
the xanthate and, if appropriate, the adjuvant is mixed with the emulsifier in substance.

2. The process as claimed in claim 1, characterized in that xanthate, if appropriate adjuvant and emulsifier are stirred with one another.

3. The process as claimed in claim 1, characterized in that xanthate, if appropriate adjuvant and emulsifier are kneaded with one another.

4. The process as claimed in one of claims 1 to 3, characterized in that for the facilitation of the mixing an excipient, in particular paraffin oil, is added.

5. An anhydrous, pharmaceutical formulation, comprising a xanthate of the formula I 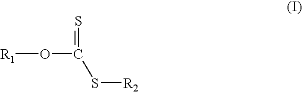
where R₁ is an optionally substituted aryl or alkyl radical and
R₂ is a metal atom, an optionally substituted alkyl, alkoxy, amino or ammonium group or halogen, and an emulsifier and, if appropriate, an adjuvant increasing the activity of the xanthate.

6. The pharmaceutical formulation as claimed in claim 5, characterized in that R₁ is a cyclododecyl, dodecyl, undecyl, decyl, tricyclo[5,2,1,0^{2,6}]decyl, nonyl, octyl, bicyclo[2,2,1]heptyl, cyclohexyl, hexyl or toluoyl radical.

7. The pharmaceutical formulation as claimed in claim 5, characterized in that R₁ is an exo/exo-tri-cyclo[5,2,1,0^{2,6}]decyl radical.

8. The pharmaceutical formulation as claimed in one of claims 5 to 7, characterized in that R₂ is a sodium or potassium atom or a dimethylglycyl ester or methyl ester group.

9. The pharmaceutical formulation as claimed in one of claims 5 to 8, characterized in that the emulsifier is a steroid or a phospholipid.

10. The pharmaceutical formulation as claimed in claim 9, characterized in that the emulsifier is selected from cholesterol, lecithin, phosphatidylserine, sphingomyelin and phosphatidylcholine.

11. The pharmaceutical formulation as claimed in one of claims 5 to 10, characterized in that 1 to 10, preferably 2 to 6, and in particular 4, parts of emulsifier are contained per one part of xanthate.

12. The pharmaceutical formulation as claimed in one of claims 5 to 11, characterized in that the adjuvant contained is an ionic detergent, preferably a fatty acid having 6 to 19 C atoms or an alkylsulfate having 8 to 18 C atoms.

13. The pharmaceutical formulation as claimed in one of claims 5 to 11, characterized in that the adjuvant contained is deoxycholic acid or a pharmaceutically tolerable salt thereof.

14. The pharmaceutical formulation as claimed in one of claims 5 to 11, characterized in that the adjuvant contained is a phosphonic acid.

15. A composition for the treatment of viral diseases, oncoses or autoimmune diseases, characterized in that it contains a pharmaceutical formulation as claimed in at least one of claims 5 to 14.

16. The composition as claimed in claim 15, characterized in that it contains tricyclo-[5,2,1,0^{2,6}]decan-9-yl xanthate as the xanthate, cholesterol or phosphatidylcholine as the emulsifier and the sodium or potassium salt of decanoic acid as the adjuvant.

17. The composition as claimed in claim 16, characterized in that it contains one part of xanthate, four parts of emulsifier and one part of adjuvant.

18. The composition as claimed in one of claims 15 to 17, characterized in that it is an ointment which contains the pharmaceutical formulation in a lipophilic substance, preferably petroleum jelly.

19. The composition as claimed in one of claims 15 to 17, characterized in that it is made available as a kit containing a physiologically tolerable medium for preparing an injection solution.

20. The composition as claimed in claim 19, characterized in that the pharmaceutical formulation or the physiologically tolerable medium contains an alkali metal hydroxide, in particular KOH.
